(19) United States
(12) Patent Application Publication
(10) Pub. No.: US 2026/0308691 A1
(43) Pub. Date: Oct. 8, 2026

(54) NOVEL USE OF AN INHIBITOR OF THE POLY-ADP RIBOSE POLYMERASE (PARP) IN THE TREATMENT OF CANCER

(71) Applicant: TESARO, Inc., Wilmington, DE (US)

(72) Inventors: Tsz cheong NG, Stevenage (GB); Anna PASTO, Stevenage (GB); Ali Raza AWAN, London (GB)

(73) Assignee: TESARO, Inc., Wilmington, DE (US)

(21) Appl. No.: 19/483,331

(22) PCT Filed: May 15, 2024
(86) PCT No.: PCT/US2024/029344

§ 371 Date: Nov. 11, 2025

(30) Foreign Application Priority Data
May 17, 2023 (GB) 2307322.4
Oct. 27, 2023 (GB) 2316472.6
Mar. 28, 2024 (GB) 240449.7

(51) Int. Cl. C12Q 1/6886; A61K 31/454

(52) U.S. Cl. CPC C12Q 1/6886; A61K 31/454; C12Q 2600/106

(57) ABSTRACT

The present invention relates to methods for personalizing cancer treatment using information obtained from patient derived organoids. Personalized treatment regimens can be identified using IC50 values (measured in organoid tumor cells alone), ratio of cell viability between tumor cells treated with a PARP inhibitor and DMSO (measured in the presence of immune cells), the length of DNA in exosomal DNA-chromatin complex identified from treatment of patient derived organoids with platinum therapy or a PARP inhibitor, or by responses to previous therapies.

### FIELD OF THE INVENTION

The present invention relates to methods for personalising cancer treatment using information obtained from patient derived organoids. Personalised treatment regimens can be identified using IC50 values (measured in organoid tumour cells alone), ratio of cell viability between tumour cells treated with a PARP inhibitor and DMSO (measured in the presence of immune cells), the length of DNA in exosomal DNA-chromatin complex identified from treatment of patient derived organoids with platinum therapy or a PARP inhibitor, or by responses to previous therapies.

### BACKGROUND TO THE INVENTION

Cancer is a serious public health problem with 562,340 people in the US dying of cancer in 2009 alone. One of the primary challenges in cancer treatment is identifying clinically useful characteristics of a patient's cancer and then, based on these characteristics, administering a treatment plan best suited to the patient's cancer.

Immune checkpoint blockade (ICB) is a promising new cancer therapy. However, to date only a small fraction of patients (ranging from 20 to 40% (Robert, 2020, Nature Communications, 11:3801)) benefit from a complete and durable response to the given immunotherapeutic therapy. This diverse response is linked to the immune infiltration difference across different types of cancer, categorized as hot (inflamed) or cold (non-inflamed), based on the rate of infiltrating immune cells and linked to the total expression levels of proteins/immune checkpoint markers.

Attempts have been made to use the hot or cold cancer phenotype and the high or low expression of immune checkpoints to stratify patients and predict the course of treatment in the clinic. This approach has met with limited success to date. This likely reflects the complexity of the immune checkpoint protein interaction network involving not only PD-1, PD-L1, CTLA4, CD80 but also other co-inhibitory receptors such as TIM3, TIGIT, CD96 and PVRIG.

Secondary cancer cell lines and xenograft models have long been used as models of cancer. However, these models are not able to predict patient outcomes to particular anti-cancer treatments, facilitating personalized medicine.

Secondary cell lines are not able to predict response to therapy, because they are highly mutated and no longer accurately reflect the primary tumour. Additionally, because of the nature of the culture, the cells miss critical intercellular interactions that would occur in the tumour microenvironment. Xenografts are more reflective of the tumour on which they are based, although the time taken to grow xenografts (typically 4-6 months) makes them incompatible with early treatment response prediction.

To overcome these obstacles, three dimensional (3D) cultures termed organoids have been established. Organoids are in vitro 3D cell aggregates, derived from primary human tumor tissues, resembling the architectural structure of the primary tissue. Indeed, organoids are generated from primary neoplastic cells embedded into a 3D basement membrane matrix (BME, Matrigel) which allows cell-to-cell and cell-to-matrix organization as in the tumour tissue. Organoids derived from multiple tumour types have been shown to retain the mutational landscape seen in the primary tumour. Importantly, the gene expression profile of organoids has been shown to be stable in organoids for 1-4 months. For this reason, organoids are sometimes referred as patient tumour's biological twin.

Organoids can be derived in the time frame of normal cancer therapy (6-9 weeks, equating to e.g. 3-4 cycles of therapy). This means that ex vivo response data from organoids can be used to personalise the treatment of the tumour used in its generation. For example, Yao and colleagues have shown that the sensitivity/resistance to chemoradiation therapies in organoids derived from primary rectal tumours correlates to response in patients in the clinic (Cell Stem Cell 2020, 26 (1): 17-26). Growth rate inhibition in organoids from metastatic colorectal cancer (mCRC) patients following irinotecan treatment has also been demonstrated to be predictive of RECIST response in mCRC patients (Ooft et al. Sci. Transl. Med., 2019, 11:513). To date, organoids represent the most promising platform to predict patient response to drug for personalized medicine (reviewed in Corsini & Knoblich, 2022, Cell, 185:2756-2769).

New methods of identifying suitable treatments for individual cancer patients are eagerly awaited.

### SUMMARY OF THE INVENTION

In a first aspect, the invention provides a method for treating cancer in a human patient wherein:
a) when the cancer is characterised in having an IC50 for a PARP inhibitor of <1.2 μM, the patient is treated with a PARP inhibitor; or
b) when the cancer is characterised in having an IC50 for a PARP inhibitor of >1.2 μM, the patient is treated with a PARP inhibitor and a PD1 or PD-L1 inhibitor.

In a second aspect, the invention provides a method for treating cancer in a human patient wherein:
a) when the cancer is characterised as having a ratio (PARP inhibitor/DMSO) of exosomal DNA length of >1.1, the patient is treated with a PARP inhibitor; or
b) when the cancer is characterised as having a ratio (PARP inhibitor/DMSO) of exosomal DNA length of <1.1, the patient is treated with a PARP inhibitor and a PD1 or PD-L1 inhibitor.

In a third aspect, the invention provides a method for treating cancer in a human patient, where the patient has previously not responded to platinum therapy, wherein the patient is treated with a PARP inhibitor and a PD1 or PD-L1 inhibitor.

In a fourth aspect, the invention provides a method for treating cancer in a human patient wherein:
a) when the cancer is characterised in having a ratio (PARP inhibitor/DMSO) in tumour cell viability of <0.75, the patient is treated with a PARP inhibitor; or
b) when the cancer is characterised in having a ratio (PARP inhibitor/DMSO) in tumour cell viability >0.75, the patient is treated with a PARP inhibitor and a PD1 or PD-L1 inhibitor.

In the context of the inventions described herein, the term PARP inhibitor refers to an inhibitor of a poly (ADP-ribose) polymerase activity. It should be noted that reference to a PARP inhibitor also includes reference to their pharmaceutically acceptable salts. In other words, “PARP inhibitor” is synonymous with “PARP inhibitor or a pharmaceutically acceptable salt thereof.

In related aspects, the invention provides methods for identifying a suitable treatment regimen for a human patient having cancer.

In a further aspect, the invention provides a general method for identifying a treatment regimen for a cancer following treatment with a test drug, comprising the steps of:
a) culturing a patient derived organoid derived from said cancer in the presence or absence of a test drug;
b) co-culturing the patient derived organoids with activated peripheral blood mononuclear cells;
c) identifying a protein selectively upregulated on infiltrating immune cells and/or on organoid tumour cells following treatment with the test drug; and
d) identifying a suitable treatment regimen as treatment with the test drug and with a modulator of the protein upregulated in c).

### DESCRIPTION OF DRAWINGS/FIGURES

FIG. 1 illustrates the multimodal assay workflow. At day 0, Patient-derived organoids (PDOs) are seeded. After 5 to 7 days (according to the proliferation rate of each sample), PDOs are treated with niraparib (1.4 μM) or DMSO (as control) for 7 days. In parallel a plate is treated with different doses of niraparib and DMSO for the cytotoxicity evaluation. At the end of the treatment, the media is collected for exosomes isolation to permit evaluation of DNA length and protein expression. Part of the PDOs undergoes RNA extraction for qRT-PCR and RNAseq analysis and the remainder is cocultured with pre activated immune cells. After 48 hours, immune phenotyping and proteomic analysis are performed by flow cytometry and multiplex IF respectively.

FIG. 2A-2B shows responses to niraparib treatment and IC50 values. A) Representative pictures of niraparib treated PDOs at the end of the treatment period. The upper panels show a niraparib resistant PDOs, whereas the lower panel show a PDOs that is more sensitive to niraparib. B) IC50 values for niraparib determined from 6 different Head and Neck (HN) and 4 different non-small cell lung cancer (Lu-P) PDOs.

FIG. 3A-3E provides information on immune cell infiltration in PDOs. A, C) Flow cytometry analysis of EpCAM and CD45 expression in a sensitive (A) or resistant (C) PDO following 1 week of DMSO or niraparib (Nir) treatment and 48 hours of coculture with immune cells. B, D) Representative pictures from multiplex imaging of sensitive (B) and resistant (D) PDOs following 1 week of treatment with DMSO or niraparib and 48 hours of coculture with immune cells (identify as CD45 positive). Scale bar 50 μm. E) Flow cytometry analysis of immune cells infiltrating DMSO or niraparib-treated PDOs; data are presented as percentage of CD45 positive cells in n=10 samples (n=6 HNSCC and n=4 NSCLC PDOs).

FIG. 4A-4B shows that CD4 positive T cells infiltrating niraparib-resistant PDOs upregulate PD1 expression. A) Flow cytometry analysis of PD1 expression on CD4 positive cells cultured alone (first peak from the bottom) or in the presence of pre-treated PDO (the second peak from the bottom indicates the immune cell subset infiltrating PDOs and the third peak from the bottom the same subset not infiltrating the PDOs). Representative plot for a niraparib (Nir) sensitive and a resistant PDO. B) Correlation plot between niraparib IC50 value and PD1 expression in CD4 positive cells infiltrating (left panel) and not infiltrating (right panel) PDOs; n=5 HNSCC and n=2 NSCLC PDOs.

FIG. 5A-5C shows PDL1 upregulation in niraparib-resistant PDOs cocultured with PBMC following niraparib treatment. A) Representative pictures from multiplex imaging analysis of a resistant PDO treated with niraparib (Nir) or control (DMSO) followed by co-culture for 48 hours with activated PBMC. Scale bar 100 μm. On the right the histogram with the mean PDL1 signal intensity analysed from all the PDOs in a slide/condition. B) Flow cytometry analysis of PDL1 expression in resistant PDOs treated with niraparib (NIR) or DMSO (as control) either cocultured with activated PBMC or left alone for 48 hours. Data are expressed as mean fluorescence intensity (MFI) from n=3 HNSCC and n=4 NSCLC PDOs. C) Correlation between PDL1 expression evaluated by flow cytometry and multiplex imaging in n=3 HNSCC and n=2 NSCLC PDOs.

FIG. 6A-6B shows that exoDNA length correlates with the niraparib IC50 in PDOs. A) Graph showing the lengths of exoDNA isolated from niraparib or DMSO-treated PDOs in 3 resistant and 3 sensitive HNSCC PDOs. Under each graph, the corresponding IC50 value. B) Graph showing the correlation between niraparib IC50 value and exoDNA length for all the 6 HNSCC (HN) and the 3 NSCLC (Lu-P) PDOs.

FIG. 7A-7C shows that PDO response to niraparib and cisplatin is similar. A-B) Dose-response curves for cisplatin (empty dots) and niraparib (gray triangles) evaluated in 4 resistant (A, 3 HNSCC and 1 NSCLC) and two sensitive (B, HNSCC) PDOs following treatment for 6 days at different drug doses (0, 0.25, 0.5, 1, 2 and 10 UM for niraparib; 0.4, 1.1, 3.3, 10 and 30 UM for cisplatin). C) Correlation between the IC50 values for cisplatin or niraparib in 5 HNSCC (HN) and 1 NSCLC (Lu-P) PDOs following 6 days of drug treatment, Spearman r 0.8857, P (two tailed) 0.033*.

FIG. 8A-8C shows that PDO response to niraparib and cisplatin is similar A) Lengths of exoDNA isolated from a representative resistant (HNX40) and sensitive (HNX29) PDOs treated with cisplatin or niraparib or controls. B) Flow cytometry analysis of CD45 positive cells infiltrating saline or cisplatin-treated PDOs; data are presented as percentage of CD45 positive cells in n=5 samples. Sample description: n=1 NSCLC classified as sensitive and n=4 (3 NSCLC and 1 HNSCC) classified as resistant according to niraparib IC50 value. C) Flow cytometry analysis of PD-L1 expression in resistant PDOs treated with cisplatin or saline solution (as control) either cocultured with activated PBMC or left alone for 48 hours. Data are expressed as mean fluorescence intensity (MFI) from n=1 HNSCC and n=3 NSCLC PDOs.

FIG. 9 shows cell viability calculated by flow cytometry analysis as the percentage of live cells within EpCAM positive cells after 7 days of treatment with 1.4 μM niraparib or DMSO (as control) and 48 h of coculture with PBMC in PDOs sensitive and resistant to naraparib. n=10 samples (n=6 HNSCC and n=4 NSCLC PDOs)

### DETAILED DESCRIPTION OF THE INVENTION

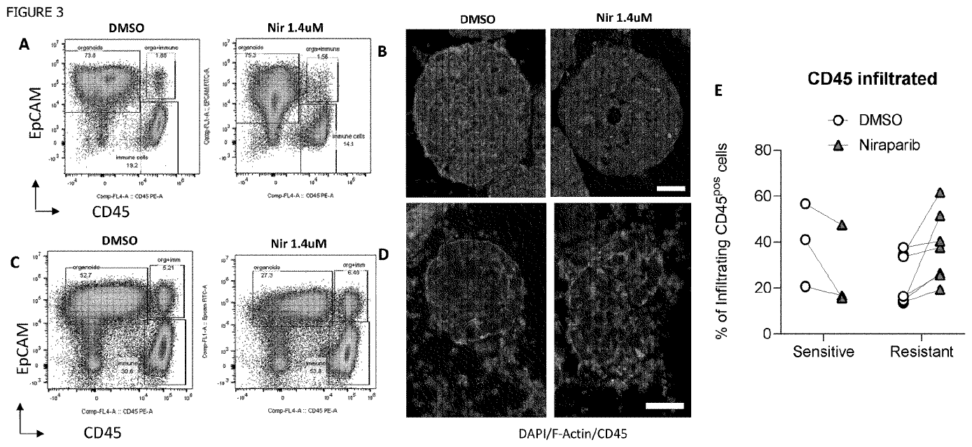

The Example and FIG. 3 shows that administration of the PARP inhibitor, niraparib, has two discrete effects upon patient derived organoids as follows:
1) A cytotoxic effect, which may be measured by the IC50.
2) An effect upon infiltration of CD45 positive cells into the patient derived organoid.

The Example and FIG. 3 shows that these effects are not independent of each other, but rather that the infiltration effect is dependent upon the cytotoxic effect such that in patients with a high IC50, niraparib treatment results in increased infiltration of CD45 positive cells and in patients with a low IC50, niraparib treatment results in decreased infiltration with CD45 positive cells. The inflection IC50 point at which increased infiltration is observed based on numerous patient derived organoids from multiple tumour types is 1.2 μM.

Whilst niraparib is well known to cause cell death by disrupting single strand break repair via the base excision pathway the mechanism by which it triggers infiltration of CD45 positive cells is less clear. Without being bound by theory however, it is possible that this effect results from cytokine or exosome release triggered by niraparib treatment. One possible explanation is that in nirparib resistant tumours, the DNA damage induced by niraparib treatment is insufficient to trigger cell death. In these cells, the damage may result in activation of the innate immune response through the release of exosomal DNA-chromatin that is sensed via the cyclic GMP-AMP Synthase—Stimulator of Interferon Genes (cGAS-STING) pathway which leads to release of type I interferon (IFN). It is therefore possible that interferon release may result in the increased infiltration of CD45 positive cells observed in FIG. 3. Even if this is not the case, type 1 interferon may be a biomarker of the changes that take place in the tumour that ultimately leads to CD45 positive cell infiltration. In addition, as discussed further below, activation of the cGAS-STING pathway may also be modified by exosomal DNA release, in a manner that is dependent on the length of the DNA in exosomes. Exosomal DNA length may thus form another biomarker of the changes that take place in the tumour that ultimately leads to CD45 positive cell infiltration.

As explained in the background to the description, high levels of T cell infiltration are associated with a “hot” cancer phenotype. The observation that niraparib treatment of patients with a high IC50 for niraparib exhibit increased levels of CD45 positive cell infiltration following niraparib treatment is evidence that this patient group may benefit from immunotherapeutic therapy. A number of patient derived organoids have been assessed and the increase or decrease in infiltrating CD45 cells was calculated from flow cytometry data. The analysis allowed us to identify the threshold ratio allowing us to discriminate sensitive from resistant samples. This threshold, which correlates with increased in CD45 positive cell infiltration and induction of the hot phenotype, was calculated to be an IC50 of 1.2 μM. Data from additional patient derived organoids may enable this threshold to be refined but it is believed that the threshold falls in the range 1-1.5 μM, more particularly 1.2-1.5 μM.

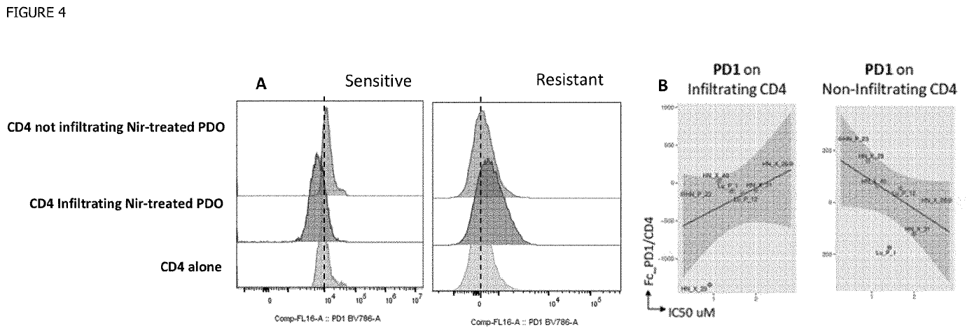

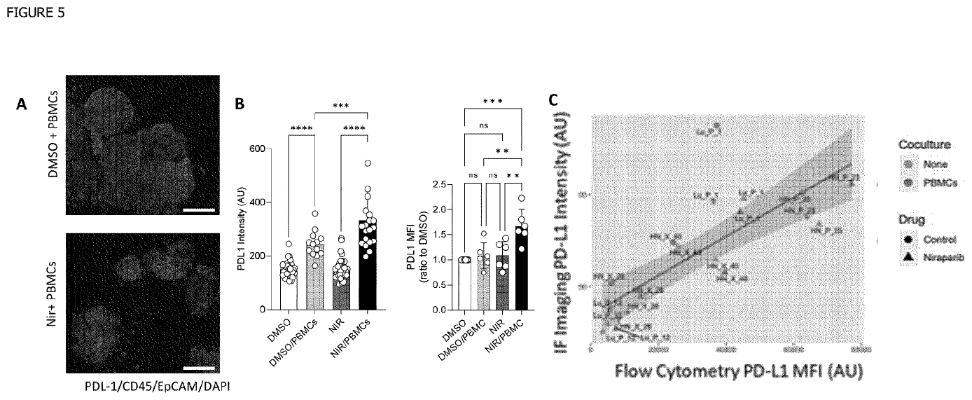

FIG. 4 shows that infiltrating CD45 positive cells exhibit high levels of PD1 expression and FIG. 5 shows that the niraparib resistant patient derived organoids exhibit high levels of PD-L1, or PD-L1 upregulation, clearly indicating a role for the PD1/PD-L1 signalling pathway in cancers which exhibit an IC50 value for niraparib of greater than the threshold level. It is believed that blockade of this signalling pathway will provide a therapeutic benefit in these types of cancers, and by extension in the patients from whom the PDOs were derived.

In view of their common mechanism of action, it is reasonable to expect that treatment of patient derived organoids with other PARP inhibitors would result in both cytotoxicity, exosomal DNA changes (which is in turn linked to the cGAS-STING pathway) and immune cell infiltration.

These observations can be exploited to identify effective personalised cancer therapies.

Accordingly, in a first aspect, the invention provides a method for treating cancer in a human patient wherein:
a) when the cancer is characterised in having an IC50 for a PARP inhibitor of <1.2 UM, the patient is treated with a PARP inhibitor; or
b) when the cancer is characterised in having an IC50 for a PARP inhibitor of >1.2 μM, the patient is treated with a PARP inhibitor and a PD1 or PD-L1 inhibitor.

In another aspect, the invention provides a PARP inhibitor for use in the treatment of cancer in a human patient wherein:
a) when the cancer is characterised in having an IC50 for a PARP inhibitor of <1.2 UM, the patient is treated with a PARP inhibitor; or
b) when the cancer is characterised in having an IC50 for a PARP inhibitor of >1.2 UM, the patient is treated with a PARP inhibitor and a PD1 or PD-L1 inhibitor.

In a further aspect, the invention provides for the use of a PARP inhibitor in the manufacture of a medicament for use in the treatment of cancer in a human patient, wherein:
a) when the cancer is characterised in having an IC50 for a PARP inhibitor of <1.2 UM, the patient is treated with a PARP inhibitor; or
b) when the cancer is characterised in having an IC50 for a PARP inhibitor of >1.2 μM, the patient is treatment with a PARP inhibitor and a PD1 or PD-L1 inhibitor.

In an alternative aspect, the IC50 threshold determining the nature of the treatment regimen is 1.0 μM. In another aspect, the IC50 threshold determining the nature of the treatment regimen is 1.1 μM. In another aspect, the IC50 threshold determining the nature of the treatment regimen is 1.3 μM. In another aspect, the IC50 threshold determining the nature of the treatment regimen is 1.4 μM. In another aspect, the IC50 threshold determining the nature of the treatment regimen is 1.5 μM.

The nature of the treatment regimen is determined by identification of the IC50 for a PARP inhibitor from a patient derived organoid. Patient derived organoids may be readily obtained using methods known in the art and indeed, services for providing patient derived organoids were commercially available at the priority date (e.g. CELLphenomics). The IC50 of the patient derived organoid for the PARP inhibitor may be identified by methods known to one of ordinary skill in the art. In one embodiment, the IC50 is identified from a dose response curve following 7 days of treatment of a patient derived organoid with a PARP inhibitor. In a more particular embodiment wherein the PARP inhibitor is niraparib, the IC50 is identified from a dose response curve following 7 days of treatment of a patient derived organoid with niraparib or a pharmaceutically acceptable salt thereof. A suitable method for determining the IC50 is provided in the Example. Whilst the Example refers to the PARP inhibitor, niraparib, an analogous method may be used to determine the IC50 of other PARP inhibitors.

In embodiments in which the cancer is characterised in having an IC50 for a PARP inhibitor of <1.2 μM (or alternative threshold), the patient is treated with a PARP inhibitor.

Suitable PARP inhibitors and treatment regimens are described further herein.

In one embodiment, the PARP inhibitor selected for treatment is the PARP inhibitor used in the IC50 calculation. For example, niraparib or a pharmaceutically acceptable salt thereof is used to treat a cancer that has an IC50 of <1.2 μM (or alternative threshold) for niraparib or a pharmaceutically acceptable salt thereof.

In embodiments in which the cancer is characterised in having an IC50 for a PARP inhibitor of >1.2 UM (or alternative threshold), the patient is treated with a PARP inhibitor and a PD1 or PD-L1 inhibitor. The PARP inhibitor and the PD1 or PD-L1 inhibitor may be administered together or separately and, when administered separately, administration may occur simultaneously or sequentially, in any order. In one embodiment, the patient is treated with a PARP inhibitor prior to treatment with a PD1 or PD-L1 inhibitor. In an alternative embodiment, the patient is treated with a PARP inhibitor and a PD1 or PD-L1 inhibitor over the same time period.

Suitable PARP inhibitors, PD1 inhibitors, PD-L1 inhibitors and treatment regimens are described further herein.

In one embodiment, the PARP inhibitor selected for treatment is the PARP inhibitor used in the IC50 calculation. For example, niraparib or a pharmaceutically acceptable salt thereof is used to treat a cancer that has an IC50 of >1.2 μM (or alternative threshold) for niraparib or a pharmaceutically acceptable salt thereof.

Suitable PD-L1 and PD1 inhibitors are described further herein.

Cancer vaccines may be used to prime the immune response to recognise tumour-specific antigens (neo-antigens) generated by a cancer. WO2020/097291 describes a method of using a personalised cancer vaccine that comprises multiple, patient/tumour specific neo-antigens to increase the number and anti-tumour activity of a patient's T-cells that recognise these neo-antigens. Additionally, the KEYNOTE-942 trial has demonstrated the clinical efficacy of this approach in stage III/IV) melanoma patients. In this trial, the use of the cancer vaccine mRNA-4157/V940 in combination with pembrolizumab (KEYTRUDA) exhibited superior efficacy as compared to pembrolizumab monotherapy.

The Example shows that PD-L1 expression is increased in cancer cells induced into the hot phenotype by administration of a PARP inhibitor. In this case, a cancer vaccine that primes the immune response to recognise PD-L1 may lead to improved therapeutic efficacy of checkpoint inhibitor therapy.

In one embodiment in which the cancer is characterised in having an IC50 for a PARP inhibitor of >1.2 μM (or alternative threshold), the patient is treated with a PARP inhibitor, a PD1 or PD-L1 inhibitor and a cancer vaccine directed to neoantigens.

In another embodiment in which the cancer is characterised in having an IC50 for a PARP inhibitor of >1.2 μM (or alternative threshold), the patient is treated with a PARP inhibitor, a PD1 or PD-L1 inhibitor and a personalised cancer vaccine directed to neoantigens generated by that cancer.

In another embodiment in which the cancer is characterised in having an IC50 for a PARP inhibitor of >1.2 μM (or alternative threshold), the patient is treated with a PARP inhibitor, a PD1 or PD-L1 inhibitor and a personalised cancer vaccine comprising mRNA4157/V940.

In one embodiment, the patient is treated with a PARP inhibitor prior to treatment with a PD1 or PD-L1 inhibitor. The skilled reader will recognise that initial treatment with a PARP inhibitor will result in CD45 positive T cell infiltration and converts the cancer to a hot phenotype. This phenotype can be recognised in patient biopsies by measurement of CD45 positive cell infiltration. Other methods of recognising the “hot” phenotype include measurement of downstream changes of the cGAS/STING pathway in patient biopsies or blood samples. In one embodiment, the hot phenotype is identified by measurement of type 1 interferon levels in patient biopsies or blood samples, particularly blood samples. In a further embodiment, the hot phenotype is identified by the measurement of PD-L1 levels in patient tumour biopsies. In a further embodiment, the hot phenotype is identified by measurement of exosomal DNA length in blood samples taken pre-and post treatment with a PARP inhibitor. More particularly, a ratio (post-treatment with a PARP inhibitor/pre treatment with a PARP inhibitor) of exosomal DNA length of <1.1 is indicative of immune sensitisation via for instance, STING activation (one of the mechanisms which can turn the tumor from cold to hot).

In one embodiment, the patient is treated with a PARP inhibitor and a cancer vaccine directed to neoantigens (more particularly, a personalised cancer vaccine directed to neoantigens generated by the cancer, and even more particularly, a personalised cancer vaccine comprising mRNA4157/V940) prior to treatment with a PD1 or PDL1 inhibitor.

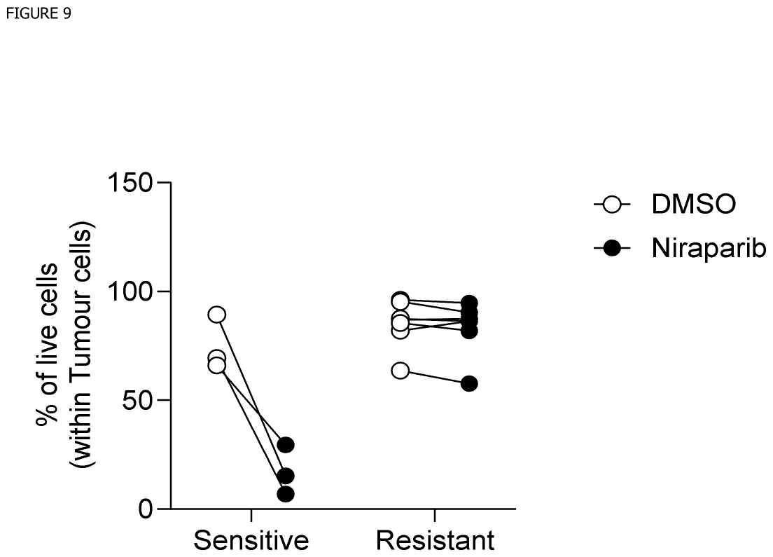

Whilst IC50 may be used to distinguish cancers that are sensitive or resistant to a PARP inhibitor and hence assign an appropriate treatment regimen, other measures taken from patient derived organoids have been found to exhibit high levels of correlation with the IC50. The skilled person would recognise that this would enable these measures to be alternatively used to distinguish the two cancer groups and assign an appropriate treatment regimen. One measure of particular note is change in cell viability between tumour cells treated with a PARP inhibitor and DMSO. This may be more reflective of the situation in vivo as it is measured in the presence of immune cells. FIG. 9 shows that the change in cell viability is only pronounced in cancers that are sensitive to the PARP inhibitor, niraparib. The relevant threshold in the change in cell viability between cancers that are sensitive or resistant to a PARP inhibitor is 0.75 i.e. the experimental cell viability is reduced by a factor of 0.75.

Accordingly, in one aspect, the invention provides a method for treating cancer in a human patient wherein:
a) when the cancer is characterised in having a ratio (PARP inhibitor/DMSO) in tumour cell viability of <0.75, the patient is treated with a PARP inhibitor; or
b) when the cancer is characterised in having a ratio (PARP inhibitor/DMSO) in tumour cell viability of >0.75, the patient is treated with a PARP inhibitor and a PD1 or PD-L1 inhibitor.

In another aspect, the invention provides a PARP inhibitor for use in the treatment of cancer in a human patient wherein:
a) when the cancer is characterised in having a ratio (PARP inhibitor/DMSO) in tumour cell viability for a PARP inhibitor of <0.75, the patient is treated with a PARP inhibitor; or
b) when the cancer is characterised in having a ratio (PARP inhibitor/DMSO) in tumour cell viability for a PARP inhibitor of >0.75, the patient is treated with a PARP inhibitor and a PD1 or PD-L1 inhibitor.

In a further aspect, the invention provides for the use of a PARP inhibitor in the manufacture of a medicament for use in the treatment of cancer in a human patient, wherein:
a) when the cancer is characterised in having a ratio (PARP inhibitor/DMSO) in tumour cell viability for a PARP inhibitor of <0.75, the patient is treated with a PARP inhibitor; or
b) when the cancer is characterised in having a ratio (PARP inhibitor/DMSO) in tumour cell viability for a PARP inhibitor of >0.75, the patient is treatment with a PARP inhibitor and a PD1 or PD-L1 inhibitor.

Like IC50, the ratio (PARP inhibitor/DMSO) in tumour cellviability of the patient derived organoid for the PARP inhibitor may be identified by methods known to one of ordinary skill in the art. In one embodiment, the ratio in tumour cell viability is determined following 7 days of treatment of a patient derived organoid with a PARP inhibitor or DMSO, then co-culture with pre-stimulated PBMCs for 48 hours. The dose of the PARP inhibitor used for the 7 days treatment is the IC30 values identified by cytotoxicity analysis. In a more particular embodiment wherein the PARP inhibitor is niraparib, the ratio in tumour cell viability is determinedfollowing7 days of treatment of a patient derived organoid with 1.4 μM niraparib or a pharmaceutically acceptable salt thereof or DMSO, then co-culture with pre-stimulated PBMCs for 48 hours. A suitable method for determining the ratio in tumour cell viability is provided in the Example. Whilst the Example refers to the PARP inhibitor, niraparib, an analogous method may be used to determine the ratio in tumour cell viability for other PARP inhibitors.

As with the IC50, due to the large gap in the ratio in tumour cell viability between the patient derived organoids that are sensitive or resistant to a PARP inhibitor, the threshold may change slightly once further patient derived organoids are available. Accordingly, in an alternative aspect, the ratio in tumour cell viability threshold determining the nature of the treatment regimen is 0.6. In another aspect, the ratio in tumour cell viability threshold determining the nature of the treatment regimen is 0.7. In another aspect, the ratio in tumour cell viability threshold determining the nature of the treatment regimen is 0.8. In another aspect, the ratio in tumour cell viability threshold determining the nature of the treatment regimen is 0.9.

The treatment regimen for a cancer characterised in having a ratio (PARP inhibitor/DMSO) in tumour cell viability of <0.75 (or alternative threshold) is the same as that described above for a cancer characterised in having an IC50<1.2 μM (or alternative threshold).

The treatment regimen for a cancer characterised in having a ratio (PARP inhibitor/DMSO) in tumour cell viability of >0.75 (or alternative threshold) is the same as that described above for a cancer characterised in having an IC50 >1.2 μM (or alternative threshold).

The Example shows that other measurements from patient derived organoids show good correlation with viability. As the skilled person will appreciate, the key aim for any measure used to identify suitable drug regimens is to maximise the number of individuals for whom the “correct” treatment regimen is selected and to minimise false positives and false negatives (in which patients are assigned to an “incorrect” treatment regimen). The numbers of false positives and false negatives can be determined from a ROC curve and Youden's index. These are conventionally referred to as the sensitivity and specificity. Table 1 shows the following variables alone are capable of predicting viability and hence assigning the patients to a particular treatment regimen:

TABLE 1

| | Pearson's | | | |
|---|---|---|---|---|
| | correlation | | | |
| | coefficient | | | |
| | (vs ratio | | | |
| | in tumour | ROC | Sensitivity | Specificity |
| Variable | cell viability) | AUC | (%) | (%) |
| IC50 | 0.56 | 1 | 100 | 100 |
| Infiltration | 0.60 | 1 | 100 | 100 |
| Exo. DNA | −0.58 | 0.91 | 100 | 67 |
| RSRD | −0.84 | 1 | 100 | 100 |
| if Ki67 | −0.69 | 1 | 100 | 100 |
| MYC | −0.64 | 0.89 | 100 | 83 |
| CD45+ TIGIT | 0.41 | 0.78 | 67 | 100 |
| CASP8 | −0.88 | 1 | 100 | 100 |
| CD4+ PD-1 | 0.087 | 0.38 | 67 | 57 |
| PD-L1 | 0.30 | 0.62 | 33 | 100 |
| CD4+ CD96 | 0.22 | 0.42 | 67 | 57 |
| polII-PCNA | −0.17 | 0.6 | 100 | 60 |

The skilled person will appreciate that measures, such as those in Table 1, that correlate well to the ratio (PARP inhibitor/DMSO) in tumour cell viability are likely to reflect changes that take place in the biological pathways for single strand break repair or in the pathways that lead to a tumour developing a “hot” phenotype. The measures may be direct or indirectly involved (i.e. causative or associative). Either way, however, individual measures can be used to assign treatment regimens with varying degress of accuracy as indicated by the sensitive and specificity analysis above. Individual measurements can be combined by machine learning to produce a score that may be a better predictor of class membership and have greater sensitivity and specificity than any individual measure. Generally, models that combine larger numbers of variables (covariates) have a greater accuracy.

The benefit to using a measure other than IC50 or viability, is that they may be more readily measured from patient samples directly, thereby potentially avoiding the need to create patient derived organoids for each cancer. The skilled person would also understand that models combining multiple covariates that are easily measured could be used as surrogates for individual measures that are “more difficult” to measure.

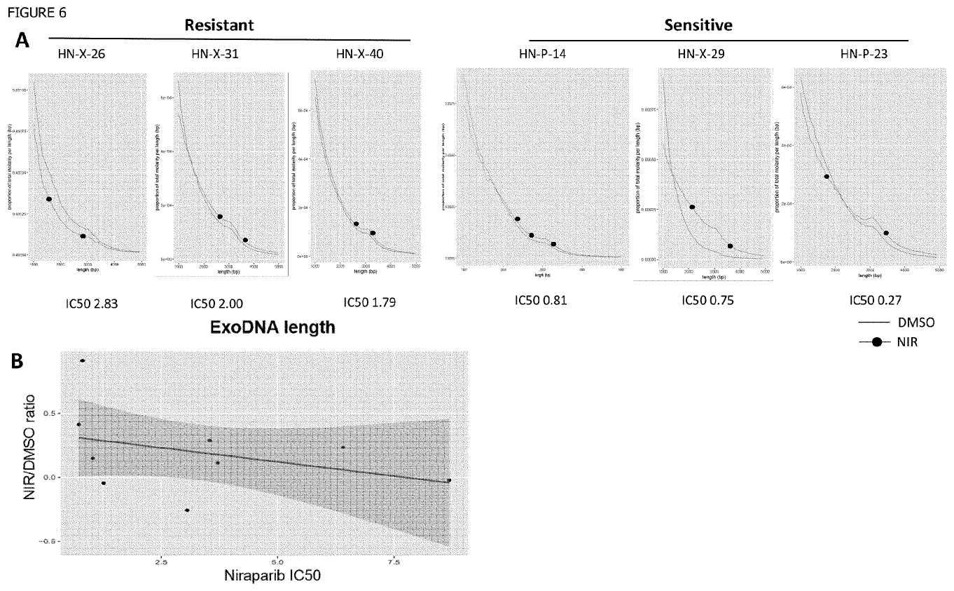

As can be seen from the table, resistance to PARP inhibition in patient derived organoids is also highly correlated to the length of DNA isolated from the exosomes (exoDNA). FIG. 6 shows that exoDNA from the PARP inhibitor, niraparib-treated patient derived organoids was shorter (compared to the exoDNA from DMSO-treated counterpart) in the patient derived organoids more resistant to the PARP inhibitor naraparib. The receiver operating characteristic (ROC) curve was calculated by plotting the ratio between exosomal DNA length in niraparib and DMSO treated patient derived organoids versus the IC50 value for 10 patient derived organoids. The analysis allowed us to identify the threshold ratio which correlates with increased in CD45 positive T cell infiltration and induction of the hot phenotype. As can be seen from the table, this was calculated to be 1.1 with a sensitivity of 100% and a specificity of 67%.

Accordingly, in one aspect, the invention provides a method for treating cancer in a human patient wherein:
a) when the cancer is characterised as having a ratio (PARP inhibitor/DMSO) of exosomal DNA length of >1.1, the patient is treated with a PARP inhibitor; or
b) when the cancer is characterised as having a ratio (PARP inhibitor/DMSO) of exosomal DNA length of <1.1, the patient is treated with a PARP inhibitor and a PD1 or PD-L1 inhibitor.

In another aspect, the invention provides a PARP inhibitor for use in the treatment of cancer in a human patient wherein:
a) when the cancer is characterised as having a ratio (PARP inhibitor/DMSO) of exosomal DNA length of >1.1, the patient is treated with a PARP inhibitor; or
b) when the cancer is characterised as having a ratio (PARP inhibitor/DMSO) of exosomal DNA length of <1.1, the patient is treated with a PARP inhibitor and a PD1 or PD-L1 inhibitor.

In a further aspect, the invention provides for the use of a PARP inhibitor in the manufacture of a medicament for the treatment of cancer in a human patient, wherein:
a) when the cancer is characterised as having a ratio (PARP inhibitor/DMSO) of exosomal DNA length of >1.1, the patient is treated with a PARP inhibitor; or
b) when the cancer is characterised as having a ratio (PARP inhibitor/DMSO) of exosomal DNA length of <1.1, patient is treated with a PARP inhibitor and a PD1 or PD-L1 inhibitor.

In these aspects, the nature of the treatment is determined by calculating a ratio of exosomal DNA length from patient derived organoids following treatment with either a PARP inhibitor or DMSO. As explained previously, patient derived organoids may be readily obtained using methods known in the art. Methods for determining exosomal DNA lengths are well known in the art. In one embodiment, exosomal DNA length is determined following 7 days of treatment of a patient derived organoid with a PARP inhibitor or DMSO. The dose of the PARP inhibitor used for the 7 days treatment is the IC30 values identified by cytotoxicity analysis. In one embodiment wherein the PARP inhibitor is niraparib or a pharmaceutically acceptable salt thereof, exosomal DNA length is determined following 7 days of treatment of a patient derived organoid with 1.4 μM niraparib or a pharmaceutically acceptable salt thereof or DMSO. A suitable method for determining the exosomal DNA length ratio is provided in the Example. Whilst the Example refers to the PARP inhibitor, niraparib, an analogous method may be used to determine the ratio for other PARP inhibitors.

The treatment regimen for a cancer characterised as having a ratio (PARP inhibitor/DMSO) of exosomal DNA length of >1.1 is the same as that described above for a cancer characterised in having an IC50<1.2 μM (or alternative threshold).

The treatment regimen for a cancer characterised as having a ratio (PARP inhibitor/DMSO) of exosomal DNA length of <1.1 is the same as that described above for a cancer characterised in having an IC50 >1.2 μM (or alternative threshold).

Because responses in patient derived organoids are predictive of patient clinical response, it is believed that the ratio (PARP inhibitor/DMSO) of exosomal DNA length in a patient derived organoid will reflect the ratio of exosomal DNA length pre and post-treatment with a PARP inhibitor. Measuring exosomal DNA length in blood samples during the course of treatment, will permit the physician to identify the point at which the tumour is converted to a “hot” phenotype and hence when therapy with a PD1 or PD-L1 inhibitor will be beneficial.

Accordingly, in one aspect, the invention provides a method for treating cancer in a human patient, wherein:
a) the patient is being treated with a PARP inhibitor; and
b) when the exosomal DNA ratio (post-treatment with a PARP inhibitor/pre treatment with a PARP inhibitor) in a blood sample from the patient is <1.1, the patient is treated with a PD1 or PD-L1 inhibitor.

In another aspect, the invention provides a PD1 inhibitor or a PD-L1 inhibitor for use in the treatment of cancer in a human patient wherein:
a) the patient is being treated with a PARP inhibitor; and
b) when the exosomal DNA ratio (post-treatment with a PARP inhibitor/pre treatment with a PARP inhibitor) in a blood sample from the patient is <1.1, the patient is treated with a PD1 or PD-L1 inhibitor.

In a further aspect, the invention provides for the use of a PD1 inhibitor or a PD-L1 inhibitor in the manufacture of a medicament for the treatment of cancer in a human patient, wherein:
a) the patient is being treated with a PARP inhibitor; and
b) when the exosomal DNA ratio (post-treatment with a PARP inhibitor/pre treatment with a PARP inhibitor) in a blood sample from the patient is <1.1, the patient is treated with a PD1 or PD-L1 inhibitor.

In a particular embodiment, the treatment regimen for patient being treated with a PARP inhibitor whose exosomal DNA ratio (post-treatment with a PARP inhibitor/pre treatment with a PARP inhibitor) is <1.1, is the same as that described above for a cancer characterised in having an IC50 >1.2 μM (or alternative threshold).

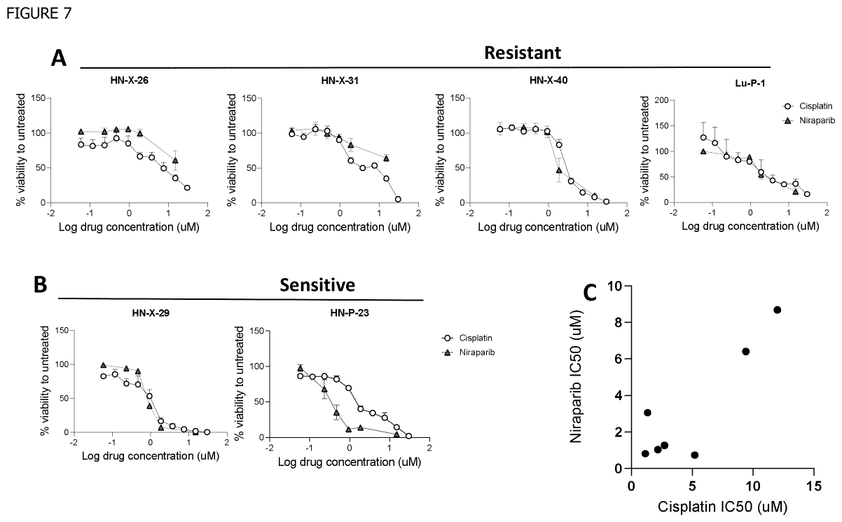

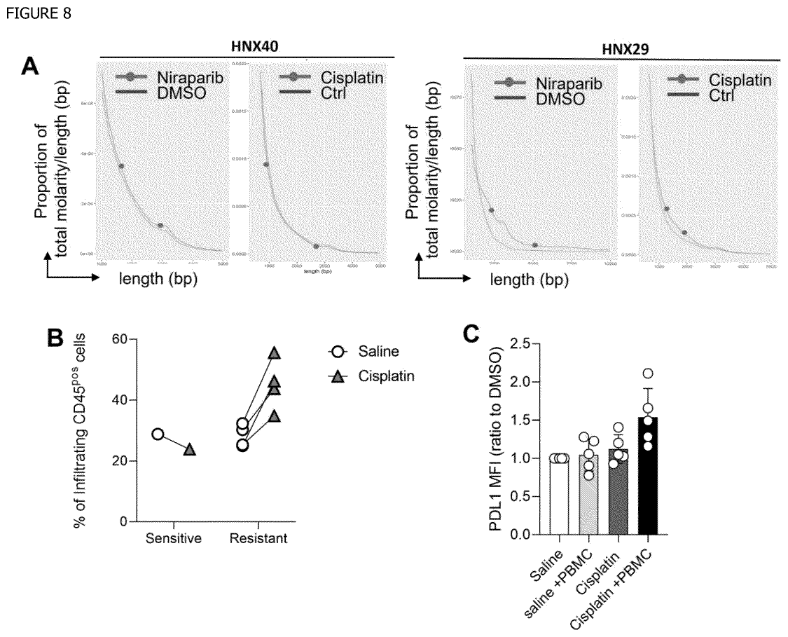

As discussed above, niraparib resistance in patient derived organoids is also observed to be highly correlated to other phenotypes that are directly observable in the patient or in patient biopsies. Because responses in patient derived organoids are predictive of patient clinical response, these associations can be used to determine whether the patient's cancer has a niraparib sensitivity of above or below the IC50 threshold of 1.2 μM and hence, permit the identification of a suitable course of treatment without the need to generate patient derived organoids. It has been observed that treatment of organoids with cisplatin results in similar effects to treatment with a PARP inhibitor. More specifically, FIGS. 7A and B shows that, in PDOs classified as resistant to niraparib treatment, cisplatin treatment resulted in increased infiltration of CD45 positive cells and in PDOs classified as sensitive, cisplatin treatment resulted in decreased infiltration with CD45 positive cells. FIG. 8 shows that exoDNA from cisplatin-treated patient derived organoids was shorter in the patient derived organoids more resistant to the PARP inhibitor naraparib. In other words, the responses in patient derived organoids to cisplatin treatment mirrored the responses to niraparib.

Example 1 and FIG. 7C demonstrates that the the IC50 values for the cisplatin and niraparib are highly correlated (p=0.033, r 0.8857) in patient derived organoids, and hence in the cancers used to generate the patient derived organoids. Accordingly, the response to platinum containing drugs such as cisplatin can be used to identify niraparib resistant cancers. As explained previously, niraparib resistant cancers may be treated stepwise with niraparib or a pharmaceutically salt thereof, followed by treatment with a PD1 or PD-L1 inhibitor.

Accordingly, in another aspect, the invention provides a method for treating cancer in a human patient, where the patient has previously not responded to platinum therapy, wherein the patient is treated with a PARP inhibitor and a PD1 or PD-L1 inhibitor.

In a further aspect, the invention provides a PARP inhibitor for use in the treatment of cancer in a human patient that has previously not responded to platinum therapy, wherein the patient is treated with a PARP inhibitor and a PD1 or PD-L1 inhibitor.

In yet another aspect, the invention provides use of a PARP inhibitor in the manufacture of a medicament for use in the treatment of cancer in a human patient that has previously not responded to platinum therapy, wherein the patient is treated with a PARP inhibitor and a PD1 or PD-L1 inhibitor.

In the context of the invention, response to platinum therapy is assessed according to RECIST 1.1 guidelines and a patient is deemed not to have responded to platinum therapy where the patient did not exhibit a complete response (CR) or partial response (PR) to the most recent round of platinum therapy.

In one embodiment, platinum therapy is treatment with an agent selected from the group consisting of: cisplatin, carboplatin, oxaliplatin, nedaplatin, triplatin tetranitrate, phenanthriplatin, picoplatin and satraplatin, optionally in combination with another chemotherapeutic agent, for example a taxane (e.g. paclitaxel or docetaxel).

The treatment regimen for a patient that has previously not responded to platinum therapy is the same as that described above for a cancer characterised in having an IC50 >1.2 μM (or alternative threshold).

A variety of variables capable of distinguishing between resistant and sensitive cancers have been discussed. These variables can be combined by machine learning to produce a score that is a better predictor of class membership than any one variable alone. Generally, models that combine larger numbers of variables (covariates) have a greater accuracy. The cut-off value that determines class membership can be determined from the ROC curve and Youden's index, and then the sensitivity and specificity of class membership can be calculated.

In all aspects described above, the cancer may be a solid tumour, for example a cytologically—or histologically-confirmed metastatic or locally advanced solid tumor. In an embodiment, the definition of cancer is also intended to include stable disease.

In particular embodiments, the cancer is selected from the group consisting of: head and neck squamous cell carcinoma (HNSCC), non small cell lung cancer (NSCLC), small cell lung cancer (SCLC), breast cancer, prostate cancer, pancreatic cancer, colorectal cancer, ovarian cancer (e.g. epilethelial ovarian cancer, or more particularly high grade serous ovarian cancer or high grade predominantly serous histology ovarian cancer), fallopian tube cancer, cervical cancer, endometrial cancer, primary peritoneal cancer, gastric cancer, kidney cancer, liver cancer, bladder cancer, skin cancer (e.g. melanoma), thyroid cancer, neuroendrocrine cancer, Ewings sarcoma, osteosarcoma, neuroblastoma, adrenocortical carcinoma, rhabdomyosarcoma and mantle cell lymphoma.

In one embodiment, the cancer is selected from the group consisting of: head and neck squamous cell carcinoma (HNSCC), non small cell lung cancer (NSCLC), small cell lung cancer (SCLC), breast cancer, ovarian cancer (e.g. epilethelial ovarian cancer, or more particularly high grade serous ovarian cancer or high grade predominantly serous histology ovarian cancer), fallopian tube cancer and primary peritoneal cancer. In a more particular embodiment, the cancer is head and neck squamous cell carcinoma (HNSCC) or non small cell lung cancer (NSCLC).

In one embodiment, the cancer is breast cancer. Breast cancer includes, but is not limited to those including ductal carcinoma in situ, lobular carcinoma, inflammatory breast cancer, invasive ductal carcinoma, Paget disease of the nipple, papillary breast cancer, medullary carcinoma, mammary carcinoma, PAM-50 classes including basal (triple-negative), HER2 positive, luminal A, luminal B and normal-like) and breast cancer in patients having a mutation in BRCA1 and/or BRCA2. In particular embodiment, breast cancer refers to triple-negative breast cancer (TNBC) or human epidermal growth factor 2 negative (HER2-) BRCA-mutated breast cancer.

In one embodiment, the cancer is ovarian cancer. Ovarian cancer includes, but is not limited to those including epithelial carcinoma; dysgerminoma, teratoma; endodermal sinus tumours; embryonal carcinoma; sex cord stromal tumours; and ovarian cancer in patients having a mutation in BRCA1 and/or BRCA2.

In an embodiment, when the PARP inhibitor is niraparib, more specifically niraparib tosylate monohydrate, the cancer may be a primary or metastatic brain cancer. In some embodiments, the primary brain cancer is selected from the group consisting of anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical Teratoid/Rhabdoid tumour, pinealoma, diffuse intrinsic pontine glioma, IDH1/2 (+) ATRX mutant glioma, malignant glioma and primitive neuroectodermal tumor of the brain. In a more particular embodiment, the primary brain cancer is a WHO grade IV tumor. In one embodiment, the primary brain cancer is glioblastoma.

In one embodiment, the cancer has previously been treated with platinum therapy.

The efficacy of cancer treatment can be measured in ways well known in the art, for example, by measurement of progression free survival (PFS), overall survival (OS), time to first subsequent therapy (TFST), time to second subsequent therapy (TSST), chemotherapy free interval (CFI), objective response rate (ORR) or by calculation of the hazard ratio for disease progression. In one embodiment, improvements in one or more of these parameters are observed following the treatment regimens outlined herein.

### PARP Inhibitor

In one embodiment, the PARP inhibitor is selected from the group consisting of niraparib, olaparib, talazoparib, rucaparib, veliparib and AZD5305 or a pharmaceutically acceptable salt thereof. The PARP inhibitor may also be selected from the group consisting of THG-008, RBN-2397, TSL-1502, NMS-03305293, HWH-340, STP06-1002, JPI-547, ABT-767, simmiparib, stenoparib, IDX-1197, SC-10914, AMXI-5001, amelparib dihydrochloride dihydrate, CK-102, IMP-4297, pamiparib, and fluzoparib or a pharmaceutically acceptable salt thereof.

AZD5305 is described in WO 2021/013735 and is known to be 5-[4-[(7-ethyl-6-oxo-5H-1,5-napthyridine-3-yl)methyl]piperazin-1-yl]-N-methyl-pyridine-2-carboxamide and has the following structure:

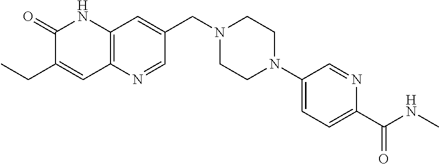 [SMILES: CCc1cc2ncc(CN3CCN(c4ccc(C(=O)NC)nc4)CC3)cc2[nH]c1=O]

In an embodiment, the PARP inhibitor is selected from the group consisting of niraparib, olaparib, talazoparib and rucaparib or a pharmaceutically acceptable salt thereof. In a further embodiment, the PARP inhibitor is selected from the group consisting of niraparib, talazoparib and rucaparib or a pharmaceutically acceptable salt thereof.

In an embodiment, the PARP inhibitor may be selective for PARP1 over PARP2.

In an embodiment, the PARP inhibitor is AZD5305 or a pharmaceutically acceptable salt thereof, as defined above.

In one embodiment, the PARP inhibitor is niraparib or a pharmaceutically acceptable salt thereof.

Niraparib free base is ((3S)-3-[4-{7-(aminocarbonyl)-2H-indazol-2-yl}phenyl]piperidine. Pharmaceutically acceptable salts of nirapraib include tosylate or a solvated or hydrated form thereof. In particular embodiments, niraparib or a pharmaceutically acceptable salt thereof refers to narparib tosylate monohydrate (((3S)-3-[4-{7-(aminocarbonyl)-2H-indazol-2-yl}phenyl]piperidinium 4-methylbenzenesulfonate monohydrate). Niraparib tosylate monohydrate can be prepared as described in Example 5 of WO2008/084261 and Example 1 of WO2009/087381.

In one embodiment, a crystalline form of niraparib tosylate monohydrate may be utilised, wherein the crystalline form is characterized by at least one X-ray diffraction pattern reflection selected from a 20 value of 9.5±0.2, 12.4±0.2, 13 2±0 2, 17.4±0.2, 18.4±0.2, 21.0±0.2, 24.9±0.2, 25 6±0 2, 26.0±0.2, and 26.9±0.2 using a Cu Long Fine Focus tube that was operated at 40 kV and 44 ma as the x-ray source. Other salts of niraparib or crystalline forms of niraparib tosylate monohydrate are known in the art and may be utilised in the treatment regimens disclosed herein. In particular, the invention contemplates use of the forms disclosed in WO 2018/183354, US20210017151, WO2020072860, WO2020072797, WO2020072796 and CN108530425.

### Treatment Regimens

The invention described herein provides for two distinct treatment regimen. One treatment regimen is administered to patients having cancers sensitive to PARP inhibition, as identified by:
i) a patient derived organoid having an IC50 for a PARP inhibitor of <1.2 μM; or
ii) having a ratio (PARP inhibitor/DMSO) in tumour cell viability of <0.75; or
iii) having a ratio (PARP inhibitor/DMSO) of exosomal DNA length of >1.1;
   wherein the patient is treated with a PARP inhibitor.

Suitable doses of the PARP inhibitor for this treatment regimen may be identified by the skilled person.

In one embodiment, the PARP inhibitor is niraparib or a pharmaceutically acceptable salt thereof. An appropriate dose of niraparib or a pharmaceutically acceptable salt thereof can be readily identified by the skilled person. In a more particular embodiment, the treatment regimen comprises treatment with an oral dose of up to 300 mg niraparib of a pharmaceutically acceptable salt thereof (weight based on free base) once daily. In some embodiments, the treatment regiment comprises an oral dose of 300 mg, 200 mg or 100 mg niraparib or a pharmaceutically acceptable salt thereof (weight based on free base) once daily. The dose of niraparib or a pharmaceutically acceptable salt thereof may be administered in one or more unit dosage forms. 100 mg capsules of niraparib are currently available, and the daily dose may be administered by providing the appropriate number of capsules.

The dose of niraparib or a pharmaceutically acceptable salt thereof may be adjusted according to clinical experience, for example in response to adverse events. Adverse events may be evaluated as per National Cancer Institute Common Terminology Criteria for Adverse Events (NCI-CTCAE), v4.03. Dose reduction in line with Table 1 should be considered for CTCAE ≥ Grade 3 treatment-related adverse reaction where prophylaxis is not considered feasible or adverse reaction persists despite treatment.

TABLE 1
Recommended dose modifications for adverse reactions

| Starting | | |
|---|---|---|
| dose level | 200 mg | 300 mg |
| First dose | 100 mg/day | 200 mg/day (two 100-mg capsules) |
| reduction | | |
| Second dose | Discontinue | 100 mg/day*(one 100-mg capsule) |
| reduction | medication. | |

In one embodiment, the PARP inhibitor is olaparib or a pharmaceutically acceptable salt thereof. An appropriate dose of olaparib or a pharmaceutically acceptable salt thereof can be readily identified by the skilled person. In a more particular embodiment, the treatment regimen comprises treatment with olaparib at a dose of 300 mg taken twice daily, equivalent to a total daily dose of 600 mg. The dose may be provided as capsules, in particular hard capsules containing 50 mg of olaparib. Therefore, to achieve a dose of 300 mg it is necessary for a patient to take six capsules. The dose may also be provided as 100 mg or 150 mg tablets, where the tablets may be film-coated.

In another embodiment, the PARP inhibitor is rucaparib or a pharmaceutically acceptable salt thereof, in particular rucaparib camsylate. In an embodiment, the treatment regimen comprises treatment with rucaparib at a dose of 600 mg taken twice daily, equivalent to a total daily dose of rucaparib of 1200 mg. The dose may be provided as tablets available as 200 mg, 250 mg and 300 mg tablets, which may be film-coated.

In another embodiment, the PARP inhibitor is talazoparib or a pharmaceutically acceptable salt thereof, in particular talazoparib tosylate. In an embodiment, the treatment regimen comprises treatment with talazoparib at a dose of 1 mg taken once daily. The dose may be provided as capsules containing talazoparib tosylate equivalent to 0.25 mg or 1 mg talazoparib.

A distinct treatment regimen is administered to patients having a cancer that is resistant to PARP inhibition as identified by:
i) a patient derived organoid having an IC50 for a PARP inhibitor of >1.2 μM; or
ii) having a ratio (PARP inhibitor/DMSO) in tumour cell viability of >0.75; or
iii) having a ratio (niraparib/DMSO) of exosomal DNA length of <1.1; or
iv) non-response to previous platinum therapy;
   wherein the patient is treated with a PARP inhibitor, a PD1 or PD-L1 inhibitor and optionally a cancer vaccine directed to neoantigens (more particularly, a personalised cancer vaccine directed to neoantigens generated by the cancer, and even more particularly, a personalised cancer vaccine comprising mRNA4157/V940).

As explained above, treatment with a PARP inhibitor induces the hot phenotype in the cancer. Suitable doses of the PARP inhibitor may be identified by the skilled person. It will be appreciated that the doses of PARP inhibitor used for conversion to the hot phenotype, may be different from the doses of PARP inhibition used in the treatment regimen for cancers sensitive to PARP inhibition.

In one embodiment, the PARP inhibitor is niraparib or a pharmaceutically acceptable salt thereof. An appropriate dose of niraparib or a pharmaceutically acceptable salt thereof can be readily identified by the skilled person. In a more particular embodiment, the treatment regimen comprises treatment with an oral dose of up to 300 mg niraparib of a pharmaceutically acceptable salt thereof (weight based on free base) once daily. In some embodiments, the treatment regiment comprises an oral dose of 300 mg, 200 mg or 100 mg niraparib or a pharmaceutically acceptable salt thereof (weight based on free base) once daily. The dose of niraparib or a pharmaceutically acceptable salt thereof may be administered in one or more unit dosage forms. 100 mg capsules of niraparib are currently available, and the daily dose may be administered by providing the appropriate number of capsules.

In one embodiment, the PARP inhibitor is olaparib or a pharmaceutically acceptable salt thereof. An appropriate dose of olaparib or a pharmaceutically acceptable salt thereof can be readily identified by the skilled person. In a more particular embodiment, the treatment regimen comprises treatment with olaparib at a dose of 300 mg taken twice daily, equivalent to a total daily dose of 600 mg. The dose may be provided as capsules, in particular hard capsules containing 50 mg of olaparib. Therefore, to achieve a dose of 300 mg it is necessary for a patient to take six capsules. The dose may also be provided as 100 mg or 150 mg tablets, where the tablets may be film-coated.

In another embodiment, the PARP inhibitor is rucaparib or a pharmaceutically acceptable salt thereof, in particular rucaparib camsylate. In an embodiment, the treatment regimen comprises treatment with rucaparib at a dose of 600 mg taken twice daily, equivalent to a total daily dose of rucaparib of 1200 mg. The dose may be provided as tablets available as 200 mg, 250 mg and 300 mg tablets, which may be film-coated.

In another embodiment, the PARP inhibitor is talazoparib or a pharmaceutically acceptable salt thereof, in particular talazoparib tosylate. In an embodiment, the treatment regimen comprises treatment with talazoparib at a dose of 1 mg taken once daily. The dose may be provided as capsules containing talazoparib tosylate equivalent to 0.25 mg or 1 mg talazoparib.

In one embodiment, the patient is treated with a PARP inhibitor prior to treatment with a PD1 or PDL1 inhibitor. In one embodiment, the time period for pre-treatment with a PARP inhibitor would be 1 week, 2 weeks, 3 weeks or 1 month. The time required could be identified by monitoring patient biopsies or blood samples for markers of the “hot” phenotype.

The treatment regimen also requires treatment with an inhibitor of PD1 or PDL1.

PD1 and PDL1 inhibitors are well known in the art and several PD1 and PDL1 inhibitors have received marketing approval. PD1 inhibitors include nivolumab, pembrolizumab, cemiplimab and dostarlimab, vopratelimab, spartalizumab, camrelizumab, sintilimab, tislelizumab, torpalimab, INCMGA00012, AMP224, AMP514 and acrixolimab. PDL1 inhibitors include atezolizumab, avelumab, durvalumab, KN035, cosibelimab, AUNP12, CA-170 and BMS-986189. Suitable doses and appropriate formulations of the PD1 or PDL1 inhibitor may readily be determined by those of skill in the art. Where the PD1 or PDL1 therapy has already received approval, the skilled person will appreciate that the dose may differ from that previously approved.

In some embodiment, the treatment regiment requires treatment with a cancer vaccine directed to neoantigens (more particularly, a personalised cancer vaccine directed to neoantigens generated by the cancer, and even more particularly, a personalised cancer vaccine comprising mRNA4157/V940). Cancer vaccines suitable for use in this invention are also known in the art. This includes mRNA-4157/V940 which is disclosed in WO2020/097291. In one embodiment, the patient is treated with a PARP inhibitor and a cancer vaccine directed to neoantigens (more particularly, a personalised cancer vaccine directed to neoantigens generated by the cancer, and even more particularly, a personalised cancer vaccine comprising mRNA4157/V940) prior to treatment with a PD1 or PDL1 inhibitor.

### Identification of Patients for Treatment

In one aspect, the invention provides a method for identifying a suitable treatment regimen for a human patient having cancer comprising:
a) calculating the IC50 for a PARP inhibitor of a patient derived organoid generated from said cancer;
b) identifying a suitable treatment regimen based on the sensitivity of the patient derived organoid to a PARP inhibitor wherein where the patient derived organoid has an IC50<1.2 μM, the suitable treatment regimen comprises treatment a PARP inhibitor, and wherein where the patient derived organoid has an IC50>1.2 μM, the suitable treatment regimen comprises stepwise treatment with a PARP inhibitor, followed by treatment with a PD1 or PD-L1 inhibitor.

In another aspect, the invention provides a method for identifying a suitable treatment regimen for a human patient having cancer comprising:
a) calculating the ratio (PARP inhibitor/DMSO) in tumour cell viability of a patient derived organoid generated from said cancer;
b) identifying a suitable treatment regimen based on the sensitivity of the patient derived organoid to a PARP inhibitor wherein where the patient derived organoid has a ratio (PARP inhibitor/DMSO) in tumour cell viability of <0.75, the suitable treatment regimen comprises treatment a PARP inhibitor, and wherein where the patient derived organoid has a ratio (PARP inhibitor/DMSO) in tumour cell viability of >0.75, the suitable treatment regimen comprises stepwise treatment with a PARP inhibitor, followed by treatment with a PD1 or PD-L1 inhibitor.

In a further aspect, the invention provides a method for identifying a suitable treatment regimen for a human patient having cancer comprising:
a) determining the ratio (niraparib/DMSO) of exosomal DNA length following treatment of a patient derived organoid with a PARP inhibitor or DMSO;
b) identifying a suitable treatment regimen based on the ratio (niraparib/DMSO) of exosomal DNA length, wherein where the patient derived organoid has a ratio (niraparib/DMSO)>1.1, the suitable treatment regimen comprises treatment with a PARP inhibitor, and wherein where the patient derived organoid has a ratio (niraparib/DMSO)<1.1, the suitable treatment regimen comprises stepwise treatment with a PARP inhibitor, followed by treatment with a PD1 or PD-L1 inhibitor.

In related aspects, the invention provides a method for treating cancer in a subject, comprising administering the suitable treatment regimen to the patient.

### Identification of Immune Blockade Therapy

The multi-modal assay described in the example permitted the identification of suitable immune blockade therapy in niraparib resistant patient derived organoids, because it specifically investigates the effects immune infiltration and antigen presentation on infiltrating immune cells. This method is capable of general application.

In one aspect, the invention provides a method for identifying a treatment regimen for a cancer following treatment with a test drug, comprising the steps of:
a) culturing a patient derived organoid derived from said cancer in the presence or absence of a test drug;
b) co-culturing the patient derived organoids with activated peripheral blood mononuclear cells;
c) identifying a protein selectively upregulated on infiltrating immune cells and/or on organoid tumour cells following treatment with the test drug; and
d) identifying a suitable treatment regimen as treatment with the test drug and with a modulator of the protein upregulated in c).

Details of suitable methods for culturing the patient derived organoids and co-culture with PBMCs are provided in the examples.

Proteins that are selectively upregulated on infiltrating immune cells (i.e. present on infiltrating immune cells at a higher level in the presence of the test drug as compared to in the absence of the test drug) or organoid tumor cells that are infiltrated by the immune cells can be identified using methods known in the art. In one embodiment, flow cytometry using antibodies against recognising candidate proteins may be used. A suitable method for conducting flow cytometry is provided in the Example. In one embodiment, the method may use an antibody recognising an immune checkpoint inhibitor protein. In one embodiment, the method may use a panel of antibodies recognising multiple proteins. In one embodiment, the panel of antibodies may recognise one or more of the following proteins: CD3, CD8, CD4, CD56, CD226, CD45, TIGIT, PD1, PDL1, EpCAM, PVRIG, TIM3, CD96, CD14 and CD11b.

In one embodiment, the modulator of the protein upregulated in c) is an inhibitor of the protein upregulated in c).

### EXAMPLE 1

### Protocol

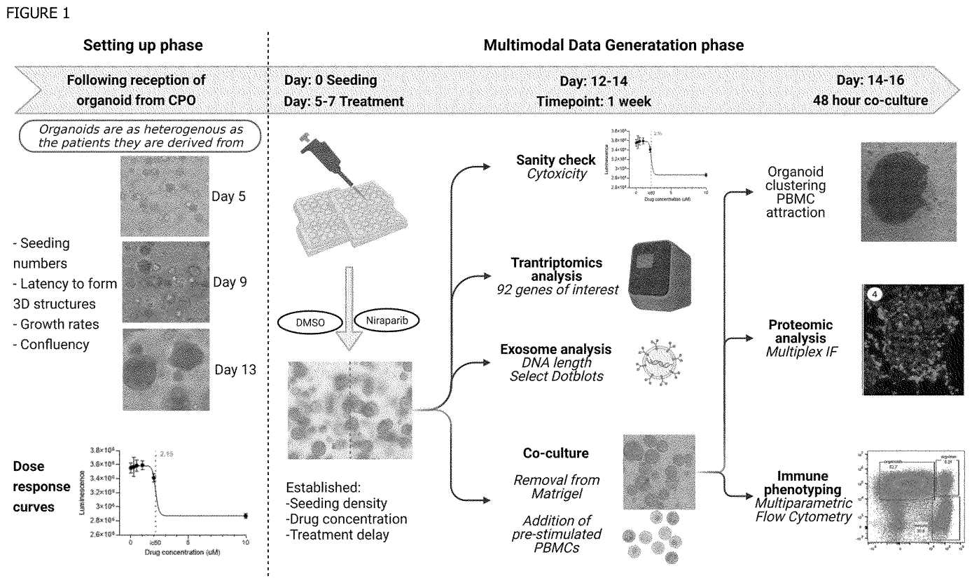

A multi-modal assay was developed to test tumour cell response to niraparib and the effects of the treatment on immune cells. The assay is illustrated in FIG. 1. FIG. 1 shows that on day 0 patient-derived organoids (PDOs) are plated 1 dome/well in 24-well plates in 15 μl of basement membrane (BME). After 5 to 7 days (according to the proliferation rate of each PDO), one plate is treated with different doses of niraparib and DMSO for cytotoxicity evaluation. The remaining plates, PDOs are treated with niraparib (1.4 μM), cisplatin (4.7 μM), DMSO or saline solution (control) for 7 days. Following the treatment period, the media from one plate/condition (i.e. one DMSO and one Niraparib-treated plate) is collected for exosomes isolation and analysis of DNA length and the PDOs are processed to extract RNA for RNAseq and qRT-PCR (see the details in the qRT-PCR analysis section). For both niraparib and cisplatin treated samples (and the respective controls) PDOs are collected from the remaining plates and, after washing out BME, are plated into low adherence 96 well plates in a 1 to 6 ratio with Peripheral Blood Mononuclear Cells (PBMCs) pre-stimulated for a total of 4 days using anti-CD3 in combination anti-CD28 (as costimulatory signal) antibodies. The PDOs are co-cultured for 48 h after which all the cells are collected, the not infiltrating immune cells are washed off and the rest is either digested to single cells or fixed and embedded as whole tissue. For flow cytometry analysis the single cells are blocked, stained with antibody mix, fixed, and run on a flow cytometer. For imaging, the embedded organoids are labelled with different antibodies, cleared, and imaged.

Protocols for conducting each of the assays forming part of the multi-modal assay are given below:

### Cytotoxicity

PDOs are seeded as single cells at a certain density (according to their growth rate) in 15 μl BME/dome.

1 dome per well in 24-wells plate with 1 ml of complete media/each well. After 5/7 days (according to the proliferation rate of the organoid), media is replaced with fresh one supplemented with 5 different doses of niraparib (10, 2, 1, 0.5 and 0.25 μM) and DMSO as controls or cisplatin (50, 25, 12.5, 6.25, 3.12, 1.56, 0.78, 0.4 and 0.19 μM) and saline solution as control. After 1 week the media is removed, and 200 μl of Cell titre glow buffer (1 X diluted in PBS) added to each well. The plate is then maintained for 1 hours in agitation (240 RPM). 150 μl of the solution is collected from each well and transferred to a 96 well plate for the reading performed with Clariostar Plus (BMG Labtech). Each dose is run in 4 replicates and the results elaborated with GraphPad Prism9 software to obtain dose/response curves. Analysis of IC50 is performed as non-linear dose response [Inhibitor]vs. response-Variable slope (four parameters).

### Viability

Cell viability is evaluated by flow cytometry analysis performed after 48 h of co-culture subsequent to 7 days of treatment with 1.4 μM Niraparib or DMSO (as control). Cells are stained with Zombie dye which enables a determination to be made of the percentage of live/dead cells within EpCAM positive cells.

### Exosome Analysis (DNA Length, DNA Sequencing and Protein Expression)

Exosomes are obtaining by a series of centrifugations and ultra-centrifugations at increasing speeds of the media collected from the PDO cultures. Each sample is split into two tubes for the ultracentrifugation (for subsequent protein and genomic analysis).

DNA is extracted from exosomes via silica-membrane based nucleic acid purification by QIAamp Blood DNA extraction Kit (QIAGEN) and quantified by Qubit dsDNA HS kit. The Genomic DNA ScreenTape assay on the Agilent 4200 Tapestation system is used to assess the length of DNA and the R package bioanalyzeR exploited to obtain the DNA size distribution and number of molecules at different sizes. Part of the exosome DNA is sequenced using Oxford Nanopore Promethion for a drug treatment and a control experiment. For both samples, reads are then aligned to the human genome (hg38) using minimap2, and the following analyses performed. For every gene for which there are any aligned reads, the following is calculated:
Fragments per kilobase million (FPKM) as a normalised metric of representation of gene in the exosome dataset
The maximum fraction of total gene length that is covered by a single nanopore read.
The median fraction of total gene length that is covered by all nanopore reads that aligned to that gene
Coding and non-coding variants (for all bases for which there was a minimum depth of 20 reads)
Hypomethylation of CpG island of coding variants

For protein evaluation, the exosomes are collected into a tube for multiparametric analysis and stained with anti-human BM2, CD80, TIM3, PDL1, CD112, LAG3, CD63, CD155, PD1, Galectin-9, S100A9, CD226, TIGIT, HMGB1, BAG6, CD96, ALIX, CD73 and P4HA1 antibodies, imaged using image stream and analyzed by image studio and IDEA software.

### RNA Analysis

For qRT-PCR analysis, cells were harvested using TripLE, followed by two washes in cold PBS and lysed in QIAzol reagent. RNA was extracted using the miRNeasy micro kit (including DNAse treatment) according to the manufacturer's instructions. The Nanodrop spectrophotometer was then used to quantify the concentration and assess the purity (260/280 ratio) of RNA recovered from cells. 500 to 1500 ng of-RNA were transcribed to cDNA using random hexamer primers and the ProtoScript II First Strand cDNA Synthesis kit, following the manufacturer's guidelines. Quantitative PCR (qPCR) reactions were set up in a 384 well plate with each well containing 10 ng of cDNA mixed with 1× PowerUp™ SYBR™ Green Master Mix and 0.25 μM of forward and reverse primers. Plates were run on the QuantStudio Flex 7 system using the standard protocol. A panel of 96 genes, including 4 housekeeping genes (HPRT1, ACTB, GAPDH and 18S), was tested for each sample. To quantify mRNA expression, CT values for each gene were normalized to the housekeeping genes by subtracting the average CT value of these.

For RNAseq, QC with an unsupervised clustering (tSNE or UMAP) or a principal component analysis (PCA) (possibly with other public cell line data) is conducted to identify any batch effects and characterize PDO transcriptome profiles. Using gene-level expression quantification (read counts or TPM) the analysis of differentially expressed genes (DEG) between niraparib treated and DMSO controls will be performed to identify significantly up—or down-regulated genes following drug treatment.

For replication stress signature analysis, starting from the data collected form RNAseq analysis, we used a reference gene signature (according to the publication McGrail et al., 2018, Cell Reports 23, 2095-2106 and Sci Trans Med 13, eabe6201, 2021) and performed a correlation analysis calculation between the reference signature (Log2FC reference) and the log 2FC of NIR-treated versus matched DMSO control PDO to elaborate RSRD variable

### Flow Cytometry Analysis

After 48 h of co-culture, PDOs are collected. The non-infiltrating immune cells are separated with a cell strainer while the organoids and infiltrating immune cells reduced to single cell level. Live dead exclusion is performed incubating the cells with Zombie dye for 30 minutes at room temperature. Following a blocking step in MACS buffer and anti-human and mouse IgG, staining is performed in

MACS buffer with mouse anti human CD3, CD8, CD4, CD56, CD226, CD45, TIGIT, PD1, PDL1, EpCAM, PVRIG, TIM3, CD96, CD14 and CD11b antibodies for one hour at 4° C. The cells are then fixed and DNA staining performed. The cell-containing tubes are running on CytoFLEX (Beckman Coulter), and data analyzed with FlowJo software.

### Multiplex Immunofluorescence Analysis

Multiplex fluorescence imaging assay is performed on a single 6-micron cryosection of fixed-frozen PDOs. PDOs from all treatment groups within the same experiment are embedded together within the same cryoblock, ensuring consistency in sectioning and staining among groups. Then target antigen staining is performed with up to four fluorophore-conjugated primary antibodies using the Ventana autostainer, immunofluorescence imaged on the Leica Cell DIVE followed by chemical inactivation of the antibody-conjugated fluorophore. The process is then repeated to include additional markers of interest. DAPI (nuclear marker) is used for the near pixel-perfect registration of images. The antibody panel includes: Ki67, p27, RPA32, yH2AX, EpCAM, CytoK, CD45, CD3, CD20, PDL1, PD1 and CD96.

We developed a new strategy for image analysis: briefly, the expression of 3 different markers (i.e. EpCAM, DAPI and CD45) were used to generate binary masks of five different organoid compartments (organoid, nuclei, cytoplasm, immune and non-immune). These masks were then used for the targeted extraction of intensity data from all images in the biomarker panel. Single mean intensity values were generated for each sample alone or in coculture with PBMC from each different treatment group and presented as heatmaps of mean fluorescence intensity for ease of comparison taking into account the proportion of each image occupied by organoids (meaning the proportion of the image that contribute to the data analysis).

### PLA Assay for R-Loop Detection

Patient-derived organoids from all treatment groups are fixed with 4% PFA for 15 min, washed in TBS buffer, incubated with 0.2% Tryton-X100 in TBS for 10 min and blocked with 5% BSA in TBS. The samples are then incubated overnight at 4 degree with the following antibodies: anti-PCNA-ab18197 (rabbit polyclonal) and anti-Pol-II-sc-56767 (mouse monoclonal) diluted in blocking solution with addition of DAPI. The day after, samples are washed, and the assay is performed as described in operational manual (Merk, DUO92013). After a final wash, coverslip are mounted with Mowiol based mounting media and samples imaged on fluorescence enabled tissue scanner (Hamamatsu, Nanozoomer). Raw images (16 bit) are analysed on ImageJ software.

### Results

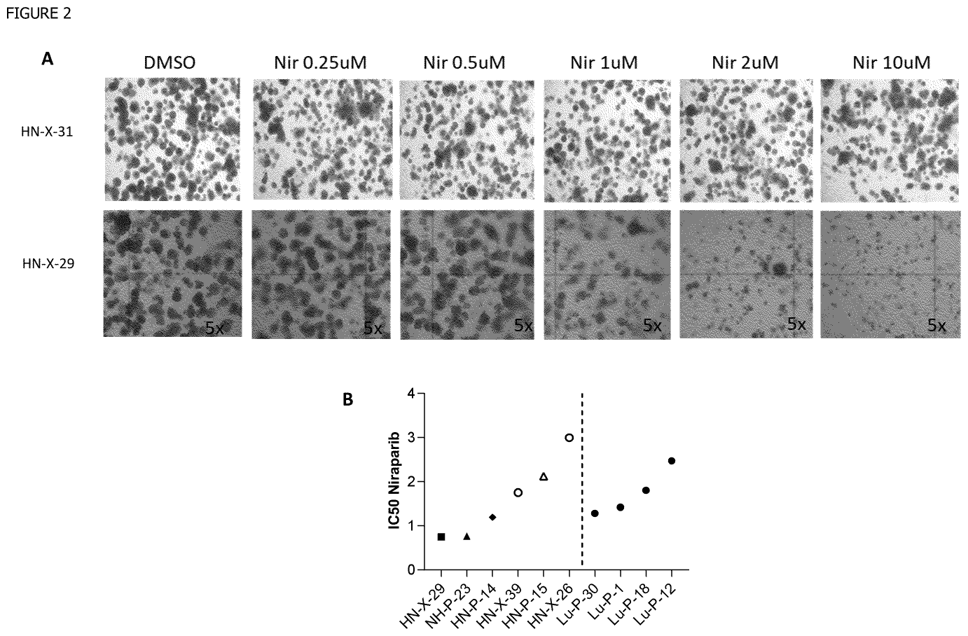

The resistance and sensitiveness to the inhibitor agent niraparib is defined by the IC50 values, calculated from a dose-response curve after 7 days of treatment. FIG. 2A shows representative pictures collected at the end of the treatment for a PDO more resistant (upper panels) or more sensitive (lower panels) to the drug. FIG. 2B shows the IC50 value for 6 different Head and Neck (HN) and 4 different non-small cell lung cancer (Lu-P) PDOs elaborated at the end of the treatment.

Results from flow cytometry (FIGS. 3A, C and E), confirmed also by imaging analysis (FIG. 3B, D), showed that tumour cell resistance to the therapy with niraparib is associated with infiltration of T cells in the TME (i.e. organoid 3D structure), suggesting a conversion of the tumour from a cold to hot phenotype. Indeed, the percentage of CD45 positive cells infiltrating the niraparib-treated PDOs increased in resistant samples (compared to DMSO) while is reduced in sensitive samples (FIG. 2E). This phenomenon of increased infiltration is observed when the IC50 value for niraparib exceed a threshold between 1-1.2 μM, whilst IC50 values below this are associated with reduced infiltration.

In the T cell fraction of PBMC infiltrating PDOs, a modulation of the immune checkpoint receptor PD1 was observede in correlation with their penetration capability within the niraparib-treated PDOs. In detail, in infiltrating CD4 positive cells, PD1 expression increases with the increased in IC50 value; on the contrary in the non-infiltrating CD4 positive cells, PD1 expression negatively correlates with niraparib IC50 value (FIG. 4).

In PDOs classified as resistant (according to the IC50 value for niraparib), we also observed upregulation of PD-L1 expression in niraparib-treated tumour cells isolated from organoids co-cultured for 48 hours with activated PBMC (FIG. 5). These results are confirmed by both multiplex imaging and flow cytometry analysis.

FIG. 9 shows that PDOs classified as resistant (according to the IC50 value for niraparib) also demonstrate a higher ratio (naraparib/DMSO) in tumour cell viability after7 days of treatment with 1.4 μM niraparib or DMSO (as control) and 48 h of coculture with PBMC.

In addition, a correlation between the length of DNA isolated from the exosomes (exoDNA) and the sensitiveness to the PARP inhibitor agent (niraparib) was identified. In fact, exoDNA from niraparib-treated PDOs was shorter (compared to the exoDNA from DMSO-treated counterpart) in the PDOs more resistant to PARP inhibitor agent. Niraparib-treated derived exoDNA length increased with the increase of niraparib sensitiveness (as shown in FIGS. 6A and B) compared to the exoDNA from DMSO treated counterpart.

In addition, the response to the agent niraparib was similar to the response to conventional chemotherapy drug cisplatin. In 6 PDOs treated for 6 days with different doses of cisplatin or naraparib, the IC50 values for the two drugs was significantly correlated (p=0.033, r 0.8857, FIG. 7C). In addition, the length of DNA isolated from exosomes collected after 6 days showed the same changes (correlating with the sensitiveness to the drug) after the two treatments. In other words, in resistant PDOs (HNX40 as representative) exoDNA length was similar or shorter after niraparib or Cisplatin treatment (compared to control) whereas was higher after both treatment in sensitive PDOs (HNX29 as representative, FIG. 8A). We also observed that tumor cells classified as resistant to niraparib treatment present higher infiltration of CD45 pos cells after cisplatin treatment (FIG. 8B). In addition, as for niraparib-treated tumor cells, we showed upregulation of PDL1 expression in cisplatin-treated tumour cells isolated from organoids co-cultured for 48 hours with activated PBMC (FIG. 8C).

Additional measurements from patient derived organoids have utility in classifying the relevant cancers as resistant or sensitive to niraparib. Table 3 lists a number of variables together with the sensitivity and specificity values determined from the ROC curve and Youden's index, and the Pearson's correlation coefficient.

TABLE 3

| | Pearson's | | | |
|---|---|---|---|---|
| | correlation | ROC | Sensitivity | Specificity |
| Variable | coefficient | AUC | (%) | (%) |
| IC50 | 0.56 | 1 | 100 | 100 |
| Infiltration | 0.60 | 1 | 100 | 100 |
| Exo. DNA | −0.58 | 0.91 | 100 | 67 |
| RSRD | −0.84 | 1 | 100 | 100 |
| if Ki67 | −0.69 | 1 | 100 | 100 |
| MYC | −0.64 | 0.89 | 100 | 83 |
| CD45+ TIGIT | 0.41 | 0.78 | 67 | 100 |
| CASP8 | −0.88 | 1 | 100 | 100 |
| CD4+ PD-1 | 0.087 | 0.38 | 67 | 57 |
| PD-L1 | 0.30 | 0.62 | 33 | 100 |
| CD4+ CD96 | 0.22 | 0.42 | 67 | 57 |
| polII-PCNA | −0.17 | 0.6 | 100 | 60 |

IC50 is measured as described above. The other variables are measured as follows:

TABLE 4

| Name | Description |
|---|---|
| Exo. DNA | The log2 fold change in the average DNA fragment length, in the |
| | range 300-50,000 base pairs, from exosomes isolated from the |
| | supernatant of the organoid model, as determined by Tape Station, |
| | between the organoid model with niraparib treatment, without |
| | immune co-culture, and the control without treatment and without |
| | immune cell co-culture. |
| if_Ki67_{—} | The difference in the total Ki67 expression as measured by |
| | immunofluorescence between the organoid model with niraparib |
| | treatment, without immune co-culture, and the control without |
| | treatment and without immune co-culture. This could be replaced |
| | by an equivalent measure from flow cytometry. |
| CD4+ PD-1 | The log2 fold change in the PD1 mean fluorescence intensity (mfi) |
| | as determined by flow cytometry, on CD4 positive cells that have |
| | infiltrated the organoid body, between the organoid model with |
| | niraparib treatment, with immune co-culture, and the control |
| | without treatment, and with co-culture. This could be replaced by |
| | an equivalent measure from immunofluorescence. |
| CD4+ CD96 | The log2 fold change in the CD96 mean fluorescence intensity (mfi) |
| | as determined by flow cytometry, on CD4 positive cells that have |
| | infiltrated the organoid body, between the organoid model with |
| | niraparib treatment, with immune co-culture, and the control |
| | without treatment, and with co-culture. This could be replaced by |
| | an equivalent measure from immunofluorescence. |
| PD-L1 | The log2 fold change in the PDL1 mean fluorescence intensity (mfi) |
| | as determined by flow cytometry, between the organoid model with |
| | niraparib treatment, with immune co-culture, and the control |
| | without treatment with co-culture. This could be replaced by an |
| | equivalent measure from immunofluorescence. |
| CASP8 | The log2 fold change in the CASP8 expression determined by PCR |
| | between the organoid model with niraparib treatment, without |
| | immune co-culture, and the control without treatment and without |
| | immune co-culture. |
| MYC | The log2 fold change in the MYC expression determined by PCR |
| | between the organoid model with niraparib treatment, without |
| | immune co-culture, and the control without treatment and without |
| | immune co-culture. |
| RSRD | The difference in genetic replication stress response signature score |
| | (as determined by analysis of gene expressions from RNAseq data) |
| | between the organoid model with niraparib treatment, without |
| | immune co-culture, and the control without treatment. |
| PolII-PCNA | The log2 fold change in the POLII-PCNA proximity ligase assay |
| | score as determined by fluorescence imaging, between the organoid |
| | model with niraparib treatment, without immune co-culture, and the |
| | control without treatment. |
| CD45+ TIGIT | The log2 fold change in the TIGIT mean fluorescence intensity (mfi) |
| | as determined by flow cytometry, on CD45 positive cells that have |
| | infiltrated the organoid body, between the organoid model with |
| | niraparib treatment, with immune co-culture, and the control |
| | without treatment, and with co-culture. This could be replaced by |
| | an equivalent measure from immunofluorescence. |
| Infiltration | The log2 fold change in the amount of CD45 cell invasion into the |
| | organoid body, as measured by the proportion of CD45 positive cells |
| | in the organoid body by flow cytometry, between the organoid |
| | model with niraparib treatment, with immune co-culture, and the |
| | control without treatment, and with co-culture. This could be |
| | replaced by an equivalent measure from immunofluorescence. |

1.-25. (canceled)

26. A method for identifying a suitable treatment regimen for a human patient having cancer comprising either:
a) calculating the ratio (PARP inhibitor/DMSO) in tumour cell viability of a patient derived organoid generated from said cancer; and
b) identifying a suitable treatment regimen based on the sensitivity of the patient derived organoid to a PARP inhibitor wherein the ratio (PARP inhibitor/DMSO) in tumour cell viability is <0.75, the suitable treatment regimen comprises treatment a PARP inhibitor, and wherein the ratio (PARP inhibitor/DMSO) in tumour cell viability is >0.75, the suitable treatment regimen comprises stepwise treatment with a PARP inhibitor, followed by treatment with a PD1 or PD-L1 inhibitor; or
a) determining the ratio (PARP inhibitor/DMSO) of exosomal DNA length following treatment of a patient derived organoid with a PARP inhibitor or DMSO; and
b) identifying a suitable treatment regimen based on the ratio (PARP inhibitor/DMSO) of exosomal DNA length, wherein the ratio (niraparib/DMSO)>1.1, the suitable treatment regimen comprises treatment with a PARP inhibitor, and wherein the ratio (PARP inhibitor/DMSO)<1.1, the suitable treatment regimen comprises stepwise treatment with a PARP inhibitor, followed by treatment with a PD1 or PD-L1 inhibitor.

27. A method according to claim 26, which comprises:
a) calculating the ratio (PARP inhibitor/DMSO) in tumour cell viability of a patient derived organoid generated from said cancer; and
b) identifying a suitable treatment regimen based on the sensitivity of the patient derived organoid to a PARP inhibitor wherein the ratio (PARP inhibitor/DMSO) in tumour cell viability of <0.75, the suitable treatment regimen comprises treatment a PARP inhibitor, and wherein the ratio (PARP inhibitor/DMSO) in tumour cell viability of >0.75, the suitable treatment regimen comprises stepwise treatment with a PARP inhibitor, followed by treatment with a PD1 or PD-L1 inhibitor.

28. A method according to claim 27, wherein the ratio in tumour cell viability is determined following 7 days of treatment of a patient derived organoid with a PARP inhibitor or DMSO, then co-culture with pre-stimulated PBMCs for 48 hours, and wherein the dose of the PARP inhibitor used for the 7 days treatment is the IC30 value identified by cytotoxicity analysis.

29. A method of treating cancer in a patient in need thereof comprising:
a. administering a PARP inhibitor to the patient, wherein the patient has a tumour cell viability ratio (PARP inhibitor/DMSO) of <0.75; or
b. administering a PARP inhibitor followed by treatment with a PD1 or PD-L1 inhibitor to the patient, wherein the patient has a tumour cell viability ratio (PARP inhibitor/DMSO) of >0.75;
wherein a tumour cell viability ratio (PARP inhibitor/DMSO) is calculated from a patient derived organoid generated from said cancer.

30. A method according to claim 29 wherein the cancer is a solid tumour.

31. A method according to claim 29, wherein the PARP inhibitor is niraparib, olaparib, talazoparib, rucaparib, veliparib, and AZD5305 or a pharmaceutically acceptable salt thereof.

32. A method according to claim 31, wherein the PARP inhibitor is niraparib tosylate monohydrate.

33. A method according to claim 26, which comprises
a) determining the ratio (PARP inhibitor/DMSO) of exosomal DNA length following treatment of a patient derived organoid with a PARP inhibitor or DMSO; and
b) identifying a suitable treatment regimen based on the ratio (PARP inhibitor/DMSO) of exosomal DNA length, wherein the patient derived organoid has a ratio (niraparib/DMSO)>1.1, the suitable treatment regimen comprises treatment with a PARP inhibitor, and wherein the patient derived organoid has a ratio (PARP inhibitor/DMSO)<1.1, the suitable treatment regimen comprises stepwise treatment with a PARP inhibitor, followed by treatment with a PD1 or PD-L1 inhibitor.

34. A method according to claim 33, wherein exosomal DNA length is determined following 7 days of treatment of a patient derived organoid with a PARP inhibitor or DMSO, and wherein the dose of the PARP inhibitor used for the 7 days treatment is the IC30 value identified by cytotoxicity analysis.

35. A method of treating cancer in a patient in need thereof comprising:
a. administering a PARP inhibitor to the patient, wherein the patient has an exosomal DNA length ratio (PARP inhibitor/DMSO) of >1.1; or
b. administering a PARP inhibitor followed by treatment with a PD1 or PD-L1 inhibitor to the patient, wherein the patient has an exosomal DNA length ratio (PARP inhibitor/DMSO) of <1.1;
wherein an exosomal DNA length ratio is determined following treatment of a patient derived organoid with a PARP inhibitor or DMSO

36. A method according to claim 35, wherein treatment with the PD1 or PD-L1 inhibitor is initiated following a measurement of exosomal DNA length in blood samples during the course of treatment.

37. A method according to claim 35 wherein the cancer is a solid tumour.

38. A method according to claim 35, wherein the PARP inhibitor is niraparib, olaparib, talazoparib, rucaparib, veliparib, and AZD5305 or a pharmaceutically acceptable salt thereof.

39. A method according to claim 38, wherein the PARP inhibitor is niraparib tosylate monohydrate.

40. A method for treating cancer in a human patient being treated with a PARP inhibitor, comprising administering a therapeutically effective amount of PD1 or PD-L1 inhibitor to the patient wherein the exosomal DNA ratio (post-treatment with a PARP inhibitor/pre treatment with a PARP inhibitor) in a blood sample from the patient is <1.1.

41. A method according to claim 40, wherein the cancer is a solid tumour.

42. A method according to claim 40, wherein the PARP inhibitor is niraparib, olaparib, talazoparib, rucaparib, veliparib, and AZD5305 or a pharmaceutically acceptable salt thereof.

43. A method according to claim 42, wherein the PARP inhibitor is niraparib tosylate monohydrate.

44. A method according to claim 40, where the cancer has previously been treated with platinum therapy.
